(19) United States
(12) Patent Application Publication
(10) Pub. No.: US 2026/0263410 A1
(43) Pub. Date: Sep. 10, 2026

(54) USE OF CALCIUM SENSING RECEPTOR ANTAGONISTS FOR TREATING OCULAR DISORDERS

(71) Applicant: THE REGENTS OF THE UNIVERSITY OF CALIFORNIA, Oakland, CA (US)

(72) Inventors: Onur CIL, San Francisco, CA (US); Neel PASRICHA, San Francisco, CA (US)

(21) Appl. No.: 19/491,385

(22) PCT Filed: Jun. 7, 2024
(86) PCT No.: PCT/US2024/033153

§ 371 Date: Dec. 8, 2025

Related U.S. Application Data
(60) Provisional application No. 63/507,430, filed on Jun. 9, 2023.

(51) Int. Cl. A61K 31/277; A61K 31/166; A61K 31/195; A61K 31/498; A61K 31/513; A61K 31/517; A61K 31/7048; A61P 27/02; A61P 27/04

(52) U.S. Cl. CPC A61K 31/277; A61K 31/166; A61K 31/195; A61K 31/498; A61K 31/513; A61K 31/517; A61K 31/7048; A61P 27/02; A61P 27/04

(57) ABSTRACT

Disclosed herein include therapy for treating ocular disorders such as dry eye disease by targeting calcium sensitive receptor (CaSR), a regulator of ocular surface ion transport.

### STATEMENT OF GOVERNMENT INTEREST

This work was supported by grants EY036139, DK126070, EY033859, and EY031372 from the National Institutes of Health. The government has certain rights in the invention.

### BACKGROUND

Tear film covers cornea and conjunctiva, thereby forming a protective barrier between external environment and the ocular surface.¹ Impaired tear production leads to dry eye disease (DED), a very common health problem particularly affecting the ageing population.^{2,3}

Tear film consists of three layers: the innermost mucin layer, the outermost lipid layer and the large aqueous layer in between. The aqueous layer of tear film primarily consists of water and electrolytes secreted by lacrimal glands, cornea, and conjunctiva and the balance between ion secretion and absorption determine tear film height.⁴

Certain ion channels and transporters are implicated in ocular surface hydration. Epithelial Na⁺ channel (ENaC) is a major pathway for Na⁺ and fluid absorption, whereas cystic fibrosis transmembrane conductance regulator (CFTR) is the major pathway for Cl⁻ and fluid secretion in the ocular surface. Due to their key roles in ocular fluid transport, ENaC and CFTR are major targets for DED drug development.^{5,6}

### BRIEF SUMMARY

Provided herein are methods of treating ocular disorders caused by reduced ocular surface hydration and/or inflammation by targeting extracellular calcium-sensing receptor (CaSR), a regulator of ocular surface ion transport. In particular, selective CaSR antagonists such as 2-chloro-6-[(2R)-3-([1,1-dimethyl-2-(2-naphthalenyl)ethyl]amino)-2-hydroxypropoxy]benzonitrile (also known as NPS-2143) are described herein as effective therapy for treating or alleviating the symptoms of DED.

According to the present disclosure, CaSR has been discovered to be prominently expressed in the cornea and conjunctiva (including in the goblet cells) of mouse and human. It is further discovered that CaSR is a key regulator of ocular surface ion transport. The effects of CaSR modulators on ocular surface ion transport were tested in mice via ocular surface potential difference (OSPD) and tear fluid volume measurements. For instance, topically administered CaSR agonist cinacalcet had no effect on baseline OSPD or Na⁺ absorption mediated by epithelial sodium channel (ENaC). However, cinacalcet concentration-dependently inhibited cAMP agonist forskolin-induced Cl⁻ secretion and CFTR activity up to 90% at 30 μM. CaSR antagonist NPS-2143 applied topically caused a large Cl⁻ secretory current in mouse ocular surface, after which forskolin had minimal secretory effect. NPS-2143 effect was reversed by CFTR inhibitor (CFTRᵢₙₕ-172), suggesting CFTR dependence of its action. Consistent with these results, single dose topical NPS-2143 treatment (0.001% or 30 μM) increased tear fluid volume by >60% for at least eight hours in mice.

Thus, CaSR antagonists offer a novel prosecretory treatment approach for ocular surface disorders (e.g., DED) by stimulating Cl⁻ and fluid secretion on the ocular surface.

Provided herein is therapeutic use of a calcium sensitive receptor (CaSR) antagonist for use in treating ocular surface disorders, including one or more dye eye diseases, keratoconjunctivitis, keratitis, or Sjogren's syndrome.

Also provided is a topical ophthalmic formulation for use in treating ocular surface disorders or increasing tear production, the ophthalmic formulation comprising a calcium sensitive receptor (CaSR) antagonist and an ophthalmically acceptable excipient.

Also provided are methods for treating ocular surface disorders or increasing tear production, the method comprising: administering to a subject in need thereof a pharmaceutical composition including a therapeutically effective amount of a calcium sensitive receptor (CaSR) antagonist and an ophthalmically acceptable excipient.

### BRIEF DESCRIPTION OF THE SEVERAL VIEWS OF THE DRAWINGS

FIG. 1. Mouse ocular surface potential difference (OSPD) measurement setup.

FIG. 2. CaSR expression in mouse ocular surface epithelia. Corneal and conjunctival sections were obtained from 12-week-old wildtype BALB/c mice. Anti-CaSR antibody (1:200 dilution) demonstrated immunofluorescence staining in the corneal and conjunctival epithelium. Hoechst 33258 was used as nucleus marker. The pictures on the right show negative controls with no primary antibody. Scale bar=25 μm.

FIG. 3. CaSR expression in human ocular surface epithelia. Corneal and conjunctival sections were obtained from human whole globes from patients with no history of visual or ocular diseases. Anti-CaSR antibody (1:200 dilution) demonstrated immunofluorescence staining in the corneal and conjunctival epithelium. Hoechst 33258 was used as nucleus marker. The pictures on the right show negative controls with no primary antibody. Scale bar=25 μm.

FIG. 4. CaSR agonist or activator cinacalcet inhibits CFTR-mediated Cl⁻ secretion in mouse ocular surface. A. Representative ocular surface potential difference (OSPD) traces in mice with control (0.2% DMSO) and various cinacalcet concentrations B. Summary of OSPD data as in A. C-F. OSPD change (Δ OSPD) induced by 100 μM amiloride (C), 10 μM forskolin (D). 10 μM CFTRᵢₙₕ-172 (E) and 100 μM ATP (F) in the presence of 0-30 μM cinacalcet in all solutions. Mean±S.E.M., n=5-15 eyes per group, Student's t-test, **p<0.01, ***p<0.001, ns: not significant.

FIG. 5. The effect of cinacalcet on ATP-induced Cl-secretion in mouse ocular surface. A. Representative ocular surface potential difference (OSPD) traces in mice with and without 30 μM cinacalcet (in all solutions) in the absence of forskolin and CFTRᵢₙₕ-172. B. Change in OSPD (Δ OSPD) induced by 100 μM ATP in the experiments in A. Mean±S.E.M., n=8 eyes per group, Student's t-test, **p<0.01.

FIG. 6. CaSR antagonist or inhibitor NPS-2143 stimulates CFTR-mediated Cl⁻ secretion in mouse ocular surface and increases tear fluid volume in mice. A. Representative ocular surface potential difference (OSPD) traces demonstrating effects of 30 μM NPS-2143, followed by 10 μM forskolin and 10 μM CFTRᵢₙₕ-172. B. Summary of OSPD data (left) and change in OSPD (Δ OSPD, right) as in A. Mean±S.E.M., n=5 eyes per group. C. Endodontic absorbent paper point tear test (EAPPTT) technique to measure tear fluid volume in awake, unanesthetized BALB/c mice. The mouse is lightly restrained and the EAPPTT is placed in the inferior fornix with forceps. D. Tear fluid volume measurements in BALB/c mice following a single 10 μL eye drop of 0.001% (30 μM) NPS-2143 or vehicle (PBS with 0.2% DMSO). n=8 eyes per group, Student's t-test, ***p<0.001 compared to the control group in the same time point.

FIG. 7. CaSR expression in mouse conjunctival goblet cells. Conjunctival sections were obtained from 12-week-old BALB/c mice. Anti-CaSR antibody (1:200 dilution) with anti-cytokeratin 7 (K7) antibody (1:200 dilution) co-staining demonstrated CaSR immunofluorescence expression in goblet cells. Hoechst 33258 was used as nucleus marker.

FIG. 8A. CaSR expression in mouse lacrimal glands. Extraorbital lacrimal gland sections were obtained from 12-week-old mice. Anti-CaSR antibody (1:200 dilution) demonstrated immunofluorescence staining throughout the lacrimal gland. Hoechst 33258 was used as nucleus marker. Scale bar=25 μm. Negative controls without anti-CaSR primary antibody confirmed the specificity of the staining.

FIG. 8B. CaSR protein expression in mouse ocular surface and lacrimal gland. Western blot was done in extraorbital lacrimal gland and conjunctiva samples from 12-week-old mice. β-actin was used as internal control in all experiments. Kidney samples from the same animals were used as positive control expressing high levels of CaSR. Primary and secondary antibodies were used at 1:1000 dilution. Representative of n=4 experiments.

FIG. 9. CaSR inhibitor NPS-2143 stimulates tear secretion in mice. (A) Standard curve of EAPPTT wetting length at different physiologic volumes of phosphate-buffered saline (PBS). n=6 per volume, R2=0.9934. (B). Tear fluid volume measurements at indicated time points in BALB/c mice following a single 10 μL eye drop of 0.001% (30 μM) NPS-2143 or 10 μL eye drop vehicle (PBS with 0.2% DMSO) at zero time. n=8 eyes per group, Student's t-test, ***p<0.001 compared to the control group in the same time point.

### DETAILED DESCRIPTION

DED is characterized with reduced ocular surface hydration that can lead to tissue damage and inflammation. As described in more detail below, targeting CaSR provides an effective therapeutical approach to treating DED by increasing fluid secretion as well as reducing inflammation.

CaSR is expressed in corneal and conjunctival epithelia Extracellular Ca²⁺-sensing receptor (CaSR) is a G-protein coupled receptor regulating various physiological processes, such as parathyroid hormone secretion, in response to changes in extracellular Ca²⁺.⁷ CaSR is expressed in various epithelial cells such as intestine and kidney, where it regulates ion transport.

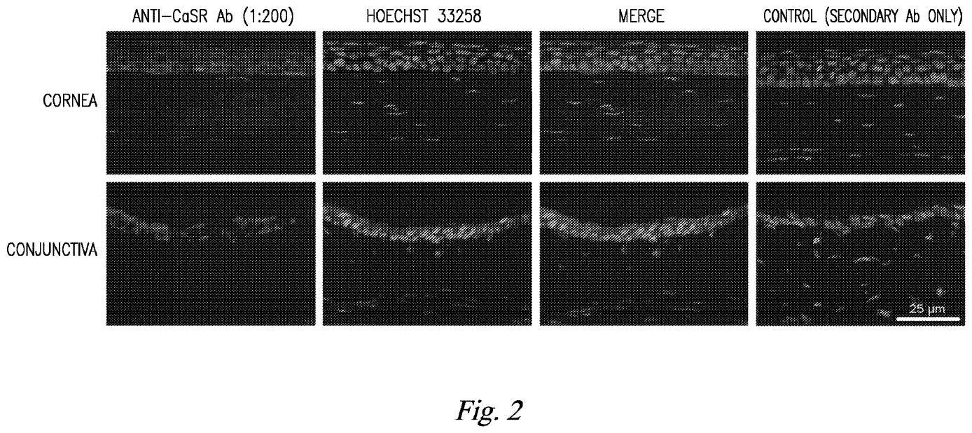

According to the present disclosure, CaSR has been found to be primarily expressed in epithelial cells of ocular surface that directly contact the tear film, based on both mice and human studies. Immunofluorescence staining in mouse cornea (FIG. 2, top) showed prominent CaSR expression in the epithelial layer with no expression in the stroma. CaSR expression was most prominent in the basal corneal epithelial layers (most of the cells had positive staining) with expression levels gradually decreasing towards the apical epithelial layers.

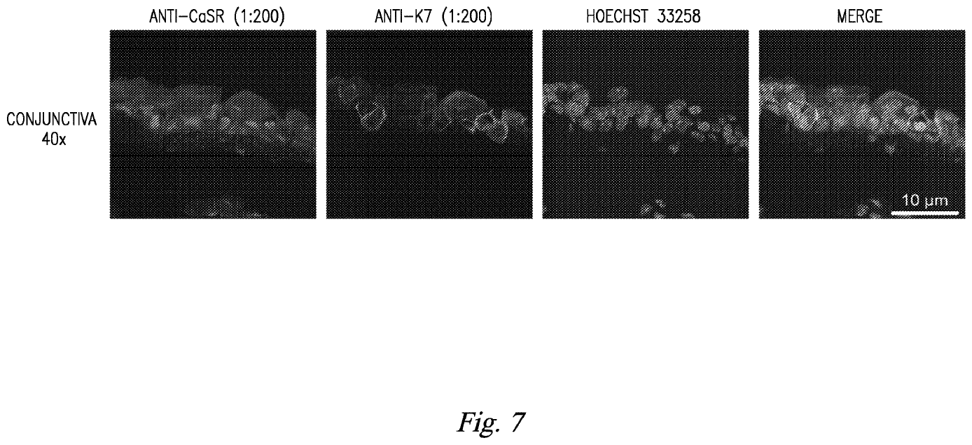

CaSR has been further found to be strongly expressed in mouse conjunctival epithelia (FIG. 2, bottom), with no expression in the fibrous layer. Additionally, CaSR was found to be expressed in mouse conjunctival goblet cells (FIG. 7).

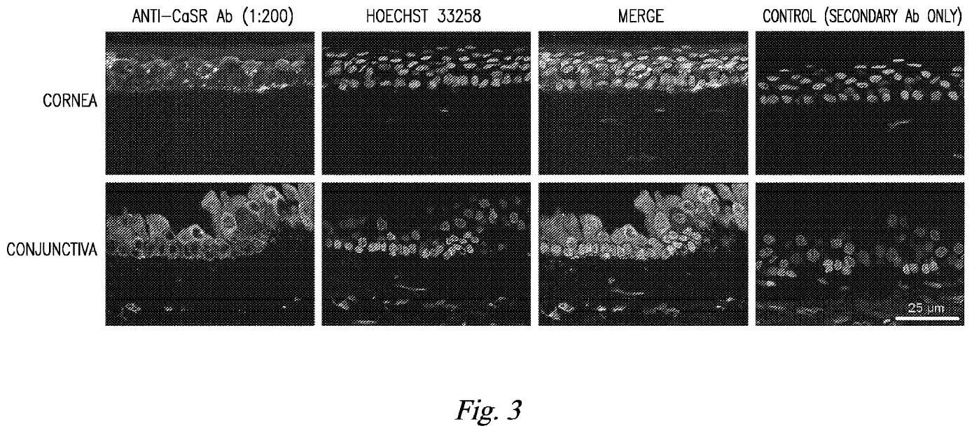

CaSR has also been found to primarily expressed in epithelial layers of human cornea and conjunctiva with minimal stromal expression (FIG. 3). Similar to mouse cornea, CaSR expression was more prominent in basal layers of human cornea with most cells expressing it.

### CaSR is Expressed in Lacrimal Glands

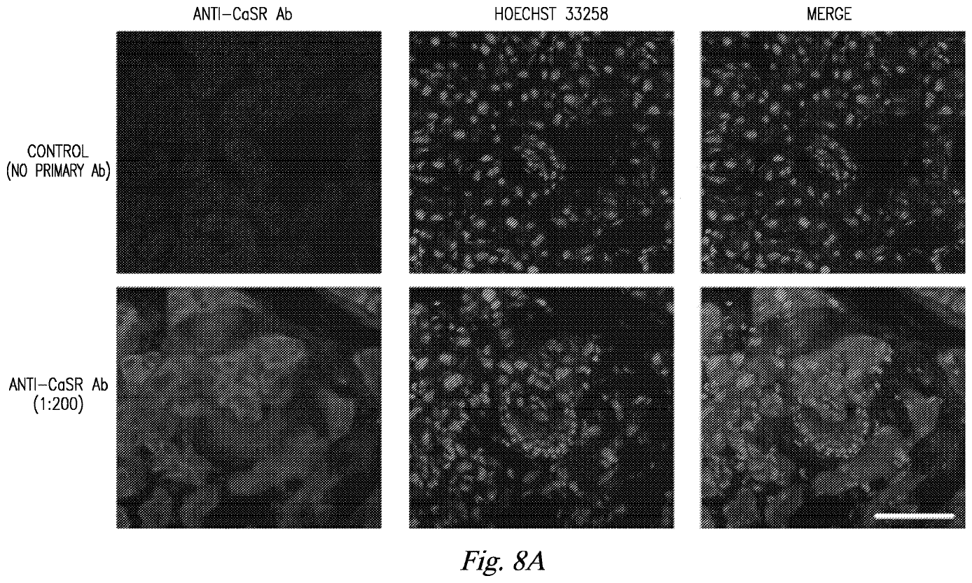

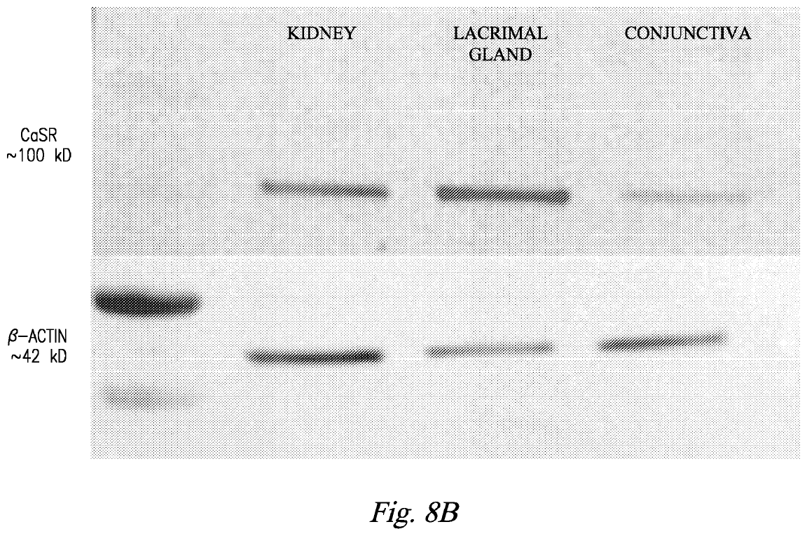

Lacrimal glands are the major source of tear fluid. According to the present disclosure, prominent CaSR expression has been found throughout the lacrimal gland following CaSR immunostaining in mouse lacrimal gland (FIG. 8A). To further confirm CaSR protein expression in lacrimal gland and ocular surface, Western blot was used and CaSR protein was found to be expressed in mouse lacrimal glands and the ocular surface. Importantly, CaSR expression was very high in mouse lacrimal glands (FIG. 8B), which was comparable to the positive control tissue kidney. These studies further validate CaSR as a therapeutic target for ocular diseases, including dry eye disease.

### CaSR is a Key Regulator of Ocular Surface Ion Transport

It is demonstrated herein, in mice studies, that CaSR plays a key role in ocular surface ion transport, namely, CaSR activation results in reduced Cl⁻ secretion, whereas CaSR inhibition stimulates CFTR-mediated Cl⁻ secretion and increases tear fluid volume.

1. CaSR Activator Cinacalcet Suppresses CFTR-Mediated Cl⁻ Secretion in Mouse ocular surface

Using pharmacological activators and inhibitors, OSPD allows studying activities of various ion channels/transporters in the ocular surface.¹² In these studies, baseline OSPD is established using a high Cl⁻ solution that mimics the tear fihn. OSPD changes induced by amiloride (ENaC inhibitor) indicates Na⁺ transport via ENaC. Similarly, OSPD changes induced by forskolin (cAMP agonist) and CFTRᵢₙₕ-172 (CFTR inhibitor) indicates Cl⁻ secretion via cAMP-activated Cl⁻ channels and particularly CFTR. Lastly, OSPD changes induced by Ca²⁺ agonist ATP indicates Cl⁻ secretion mediated by CaCCs.

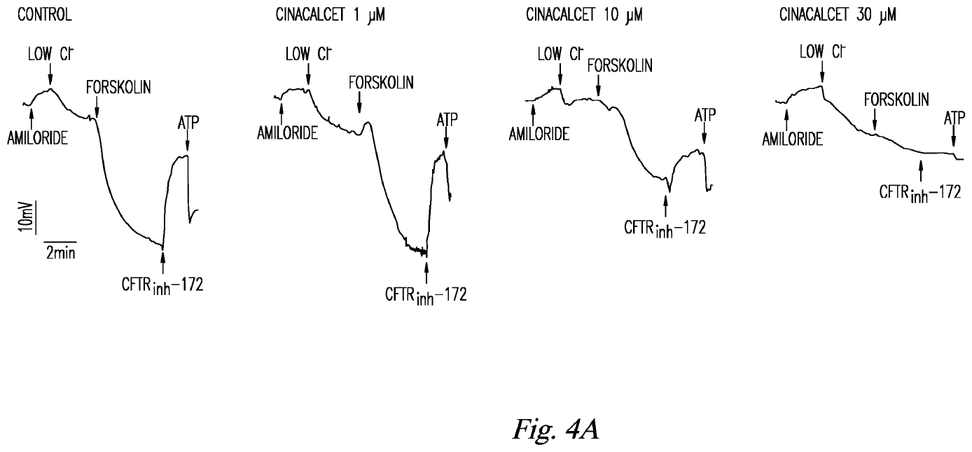

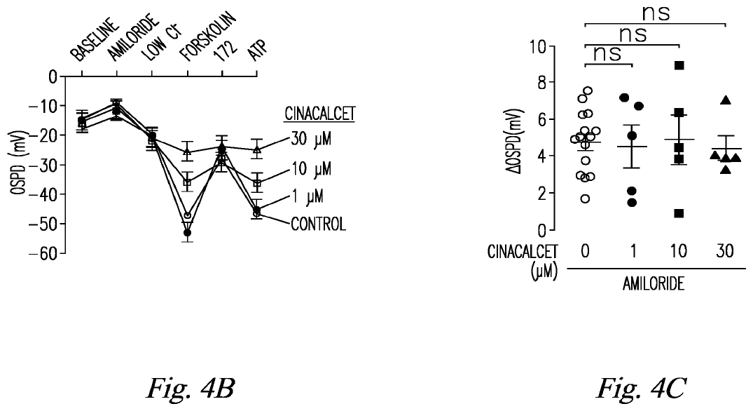

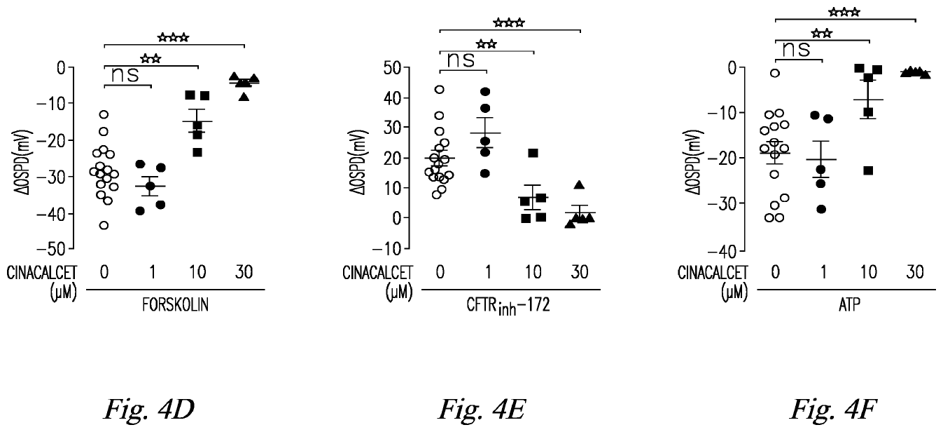

Sequential perfusion of the ocular surface with solutions containing amiloride (ENaC inhibitor), forskolin (cAMP agonist), CFTRᵢₙₕ-172 (CFTR inhibitor), and ATP (Ca²⁺ agonist) thus allows determining activities of ENaC, CFTR, and CaCC ion channels, respectively (FIG. 4A). Cinacalcet treatment had no effect on ENaC activity as suggested by lack of its effect on amiloride-induced depolarization (FIGS. 4A-C). However, cinacalcet concentration-dependently inhibited CFTR activity as suggested by up to 90% lower forskolin (FIG. 4D) and CFTRmh-172 (FIG. 4E) responses at 30 μM. In this setting, cinacalcet treatment similarly suppressed CaCC activity, as suggested by 90% lower ATP-induced hyperpolarization (FIG. 4F). The results suggest that CaSR activation (e.g., by cinacalcet) inhibits CFTR-mediated Cl⁻ secretion in the ocular surface but having no effects on ENaC-mediated Na²⁺ absorption.

### 2. Cinacalcet Suppresses CaCC-Mediated Cl⁻ Secretion in Mouse Ocular Surface

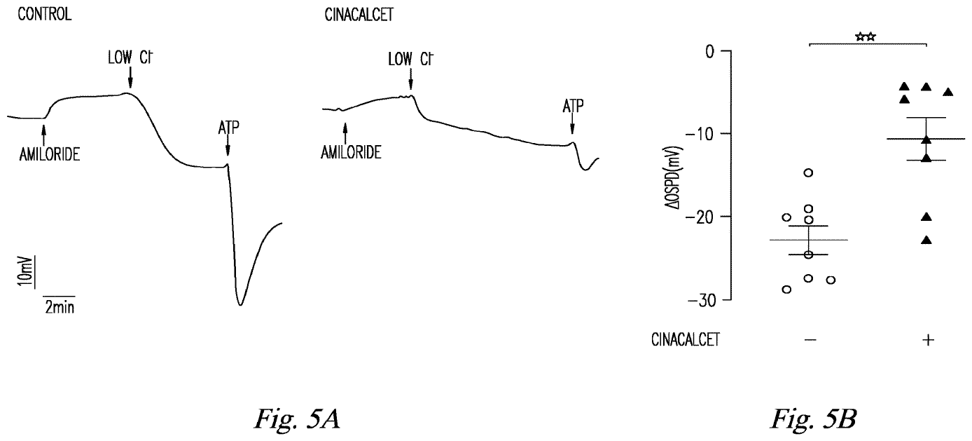

Although CFTR and CaCC are activated by different intracellular signaling pathways (cAMP and Ca²⁺, respectively), the cross talk between intracellular cAMP and Ca²⁺ signaling pathways might cause confounding effects when activators or inhibitors of these pathways are sequentially applied in the same system.¹⁶ OSPD experiments without prior forskolin and CFTRᵢₙₕ-172 treatments demonstrated the effects of cinacalcet on CaCC directly. In this setting, cinacalcet still inhibited ATP response by ~50% (FIG. 5), albeit at a lower effect than the experiments where ATP was added after forskolin and CFTRᵢₙₕ-172 (compare with FIG. 4F). These results suggest that CaSR activator cinacalcet also inhibits CaCC-mediated Cl⁻ secretion in the ocular surface.

### 3. CaSR Antagonist NPS-2143 Induces CFTR-Mediated Cl⁻ Secretion in Mouse Ocular Surface

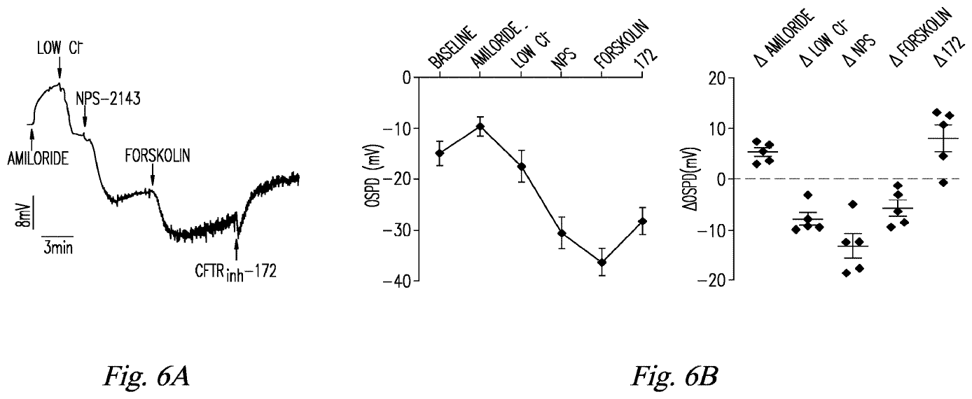

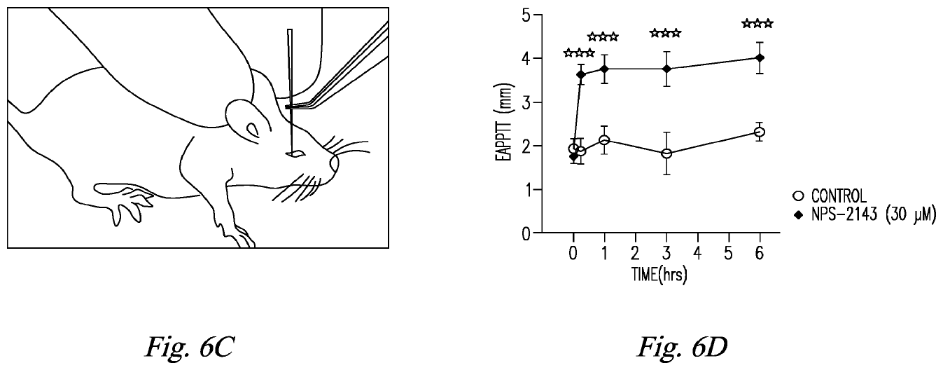

Tear fluid contains 0.4-1.1 mM Ca²⁺ which is comparable to plasma ionized Ca²⁺.¹⁷ It is hypothesized that physiological Ca²⁺ concentrations in tear fluid have tonic antisecretory effects in the ocular surface due to CaSR activation. Using CaSR antagonist NPS-2143 in the OSPD experiments confirmed the hypothesis. Perfusion of ocular surface with 30 μM NPS-2143 led to large hyperpolarization (−13 mV), which was 70% of subsequently applied maximal forskolin-induced hyperpolarization after which cAMP agonist forskolin had minimal further secretory effect (FIGS. 6A and 6B). NPS-2143 and forskolin responses were partially reversed by CFTRᵢₙₕ-172 suggesting CFTR dependence of its effect. These results suggest that CaSR has sustained antisecretory effects in the ocular surface via inhibition of CFTR activity.

### 4. NPS-2143 Increases Tear Fluid Volume in Mice and its Effect is Reversible

The effects of NPS-2143 on tear fluid volume were tested by EAPPTT in mice that were awake. Topically administered NPS-2143 (0.001% or 30 μM, single 10 μL dose) caused 60% increased tear fluid volume in mice as early as 15 minutes after treatment. Importantly, the effect of single dose NPS-2143 lasted at least 6 hours (FIG. 6D).

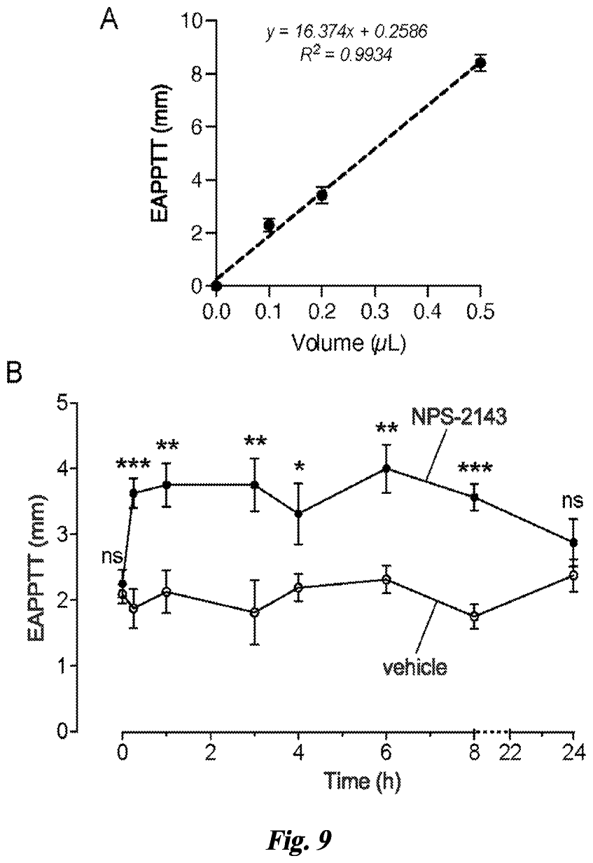

The NPS-2143 effect on tear volume is durable and reversable. In another test by EAPPTT, the accuracy of EAPPTT was confirmed by a standard curve (FIG. 9A). Topically administered NPS-2143 (0.001% or 30 μM, single 10 μL dose) caused ~60% increased tear fluid volume in mice as early as 15 minutes after treatment (EAPPTT wetting length 2.3±0.2 mm at baseline versus 3.6±0.2 mm 15 minutes after NPS-2143 administration, mean±SEM, p<0.001). The effect of single dose NPS-2143 lasted at least 8 hours (EAPPTT wetting length 3.6±0.2 mm 8 hours after NPS-2143 administration) compared to the vehicle group. Importantly. NPS-2143 effect was reversible, as suggested by the return of tear volume to near baseline values 24 h after treatment (FIG. 9B). These results suggest that topical treatment with CaSR antagonists can increase tear fluid volume and the potential sustained efficacy of NPS-2143 as eye drops administered 2-3 times per day.

### Therapeutic Use of CaSR Antagonists for Treating Ocular Surface Disorders

According to the present disclosure, CaSR is prominently expressed in the ocular surface, including corneal and conjunctival epithelia of mice and humans. In particular, CaSR is expressed in conjunctival goblet cells, which suggests that CaSR may also play a role in mucin production and/or secretion. CaSR is additionally expressed in lacrimal glands, the major source of tear fluid.

According to the present disclosure, CaSR antagonists or inhibitors have prosecretory effects on the ocular surface and potentially on the lacrimal glands as well.

Given their prosecretory effects, CaSR antagonists can be effective for treating ocular surface disorders, e.g., dry eye disease, by increasing tear volume and/or reducing inflammation, in dry eye produced by multiple etiologies, including disorders targeting lacrimal glands such as Sjogren's syndrome.

“Dry eve disease” or DED refers to a disease in which a patient experiences dryness in one or both eyes. Dry eye disease is marked by an insufficient quality or quantity of tear production. Exemplary symptoms of dry eye disease include irritation, burning, stinging, discharge, foreign body sensation, tearing, blurred vision, or a combination of two or more symptoms. Dry eye disease may alternatively be referred to as dry eye syndrome, keratoconjunctivitis sicca, dysfunctional tear syndrome, or lacrimal keratoconjunctivitis. Dry eye disease may be caused by multiple etiologies, including medications, advanced age, rosacea, blepharitis, autoimmune disorders (e.g., Sjogren's syndrome), inflammations (e.g., keratoconjunctivitis, and keratitis), diabetes, thyroid disorders, Vitamin A deficiency, environmental conditions (e.g., dry or windy enviromnents), seasonal allergies, sun exposure, or laser eye surgery. In embodiments, dry eye disease may be diagnosed by Schirmer's tear tests and/or ocular surface staining patterns of Lissamine green, Rose Bengal, and/or fluorescein dyes.

“Increasing tear production” or “increasing tear volume” refers to increasing the tear production in a patient relative to a control. The control can be the same patient prior to treatment, a statistical group of patients who have not been treated, or a different patient who has not been treated. In various embodiments, increasing tear production refers to doubling the tear production of the patient when compared to the tear production of the patient prior to treatment (or when compared to another control) with the active agents described herein. In other embodiments, increasing tear production refers to tripling or quadrupling tear production when compared to the tear production of the patient prior to treatment (or when compared to another control) with the active agents described herein. In embodiments, increasing tear production refers to increasing the tear production of a patient to within a normal range of tear production for the patient relative to a control or to applicable standards known in the art, and include, for example, Schirmer's tear tests I (unanesthetized) and II (anesthetized, measured after instillation of topical 0.5% proparacaine).

As used herein, “CaSR antagonists” or “CaSR inhibitors” effective for the dry eye treatment according to the present disclosure include any compounds or agents that exhibit IC₅₀ of 10 micromolar or lower against CaSR. Examples of CaSR antagonists, without limitations, NPS-2143, NPS-2390, NPSP-795 (or SB-423562), ronacaleret, encaleret (JTT-305), calcium-sensing receptor antagonists I, Calhex 231, ligustroflavone, SB-423557, CaSR antagonist-1. Table 1 below shows the chemical names and structures of these examples.

TABLE 1

| Common Name | Structure | Chemical Name |
|---|---|---|
| NPS-2143 | 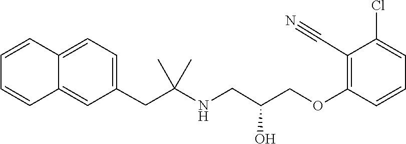 [SMILES: CC(C)(Cc1ccc2ccccc2c1)NC[C@@H](O)COc1cccc(Cl)c1C#N] | 2-chloro-6-[(2R)-3-([1,1- dimethyl-2-(2- naphthalenyl)ethyl]amino)-2- hydroxypropoxy benzonitrile |
| NPS-2390 | 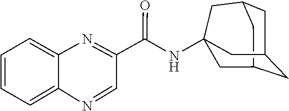 [SMILES: O=C(NC12CC3CC(CC(C3)C1)C2)c1cnc2ccccc2n1] | N-((3s,5s,7s)-adamantan-1- yl)quinoxaline-2-carboxamide |
| NPSP-795 SB-423562 | 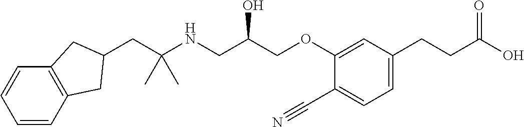 [SMILES: CC(C)(CC1Cc2ccccc2C1)NC[C@@H](O)COc1cc(CCC(=O)O)ccc1C#N] | (R)-3-(4-cyano-3-(3-((1-(2,3- dihydro-1H-inden-2-yl)-2- methylpropan-2-yl)amino)-2- hydroxypropoxy)phenyl) propanoic acid |
| Ronacaleret | 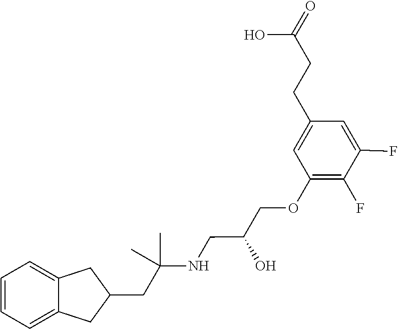 [SMILES: CC(C)(CC1Cc2ccccc2C1)NC[C@@H](O)COc1cc(CCC(=O)O)cc(F)c1F] | (R)-3-(3-(3-((1-(2,3-dihydro- 1H-inden-2-yl)-2- methylpropan-2-yl)amino)-2- hydroxypropoxy)-4,5- difluorophenyl)propanoic acid |
| Encaleret (JTT- 305) | 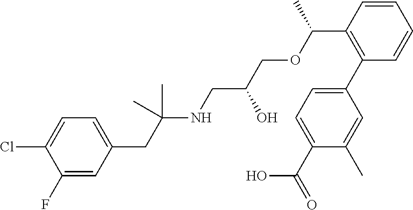 [SMILES: Cc1cc(-c2ccccc2[C@@H](C)OC[C@H](O)CNC(C)(C)Cc2ccc(Cl)c(F)c2)ccc1C(=O)O] | 2′-((R)-1-((R)-3-((1-(4-chloro- 3-fluorophenyl)-2- methylpropan-2-yl)amino)-2- hydroxypropoxy)ethyl)-3- methyl-[1,1′-biphenyl]-4- carboxylic acid |
| Calcium- Sensing Receptor Antagonists I | 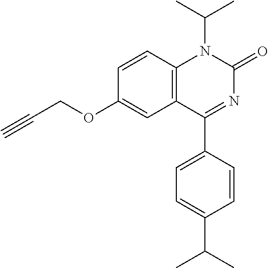 [SMILES: C#CCOc1ccc2c(c1)c(-c1ccc(C(C)C)cc1)nc(=O)n2C(C)C] | 1-isopropyl-4-(4- isopropylphenyl)-6-(prop-2-yn- 1-yloxy)quinazolin-2(1H)-one |
| Calhex 231 | 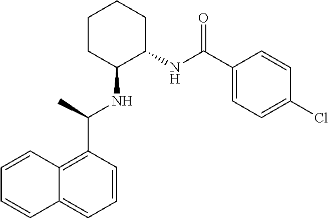 [SMILES: C[C@@H](N[C@H]1CCCC[C@@H]1NC(=O)c1ccc(Cl)cc1)c1cccc2ccccc12] | 4-chloro-N-((IS,2S)-2-(((R)-1- (naphthalen-1- yl)ethyl)amino)cyclohexyl) benzamide |
| Ligustroflavone | 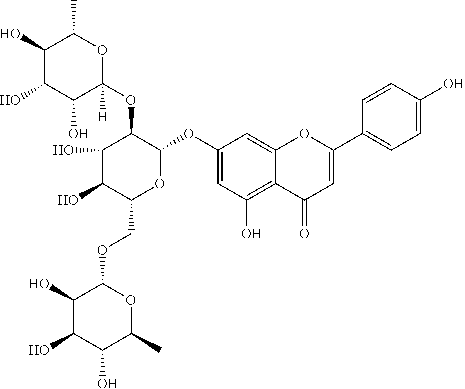 [SMILES: C[C@@H]1O[C@@H](OC[C@H]2O[C@@H](Oc3cc(O)c4c(=O)cc(-c5ccc(O)cc5)oc4c3)[C@H](O[C@@H]3O[C@@H](C)[C@H](O)[C@@H](O)[C@H]3O)[C@@H](O)[C@@H]2O)[C@H](O)[C@H](O)[C@H]1O] | 7-(((2S,3R,4S,5S,6R)-4,5- dihydroxy-3- (((2S,3R,4R,5R,6S)-3,4,5- trihydroxy-6-methyltetrahydro- 2H-pyran-2-yl)oxy)-6- ((((2R,3R,4R,5R,6S)-3,4,5- trihydroxy-6-methyltetrahydro- 2H-pyran-2- yl)oxy)methyl)tetrahydro-2H- pyran-2-yl)oxy)-5-hydroxy-2- (4-hydroxyphenyl)-4H- chromen-4-one |
| SB-423557 | 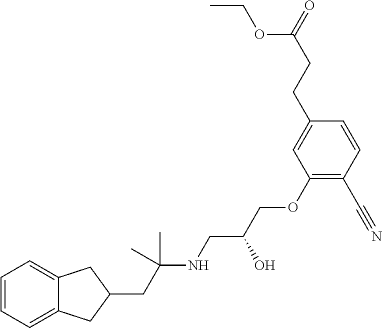 [SMILES: CCOC(=O)CCc1ccc(C#N)c(OC[C@H](O)CNC(C)(C)CC2Cc3ccccc3C2)c1] | ethyl (R)-3-(4-cyano-3-(3-((1- (2,3-dihydro-1H-inden-2-yl)-2- methylpropan-2-yl)amino)-2- hydroxypropoxy)phenyl) propanoate |
| CaSR antagonist-1 | 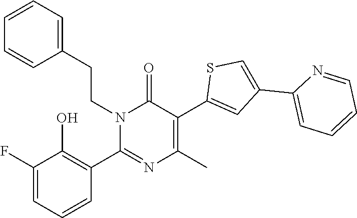 [SMILES: Cc1nc(-c2cccc(F)c2O)n(CCc2ccccc2)c(=O)c1-c1cc(-c2ccccn2)cs1] | 2-(3-fluoro-2-hydroxyphenyl)- 6-methyl-3-phenethyl-5-(4- (pyridin-2-yl)thiophen-2- yl)pyrimidin-4(3H)-one |

Currently approved DED drugs primarily target inflammation and do not correct tear fluid volume or hyperosmolarity, which are the major drivers of pathology.¹⁸⁻²¹ Several prosecretory or anti-absorptive DED therapies targeting ion transport are currently in clinical development such as small-molecule CFTR activators.^{5,6,22-25} Diquafosol is a purinergic P2Y2 receptor agonist that primarily works by increasing CaCC activity in the ocular surface. Although it is approved in Japan, diquafosol did not meet the primary endpoints in clinical trials and is not approved by FDA for DED. Another drawback with diquafasol is that it requires frequent administration (six times a day), although a long-acting formulation is currently in clinical development.²⁶

In contrast to the conventional DED therapy, CaSR antagonist, such as NPS-2143, has sustained prosecretory effect in mice for at least 8 hours after single dose treatment. In addition to their prosecretory effects as described here, CaSR antagonists were shown to have anti-inflammatory effects in other tissues.²⁷

Thus, one embodiment provides a method for treating DED comprising topically administering to a subject in need thereof a pharmaceutical composition including a CaSR antagonist and an ophthalmically acceptable excipient.

Suitable CaSR antagonists may be any of the calcilytic drugs. Calcilytic drugs are typically orally administered and cause a rapid increase in plasma levels of PTH accompanied by a rise in plasma levels of Ca²⁺. In more specific embodiments, the CaSR antagonist is NPS-2143, ronacaleret or encaleret. In other more specific embodiments, the CaSR antagonist is NPS-2390, NPSP-795 (or SB-423562), calcium-sensing receptor antagonists I, Calhex 231, ligustroflavone, SB-423557, or CaSR antagonist-1. Additional examples of the calcilytic drugs are described in, for example, Nemeth E. F. et al, Calcif Tissue Int 98:341-358 (2016).

CaSR antagonists and pharmaceutical compositions described herein are to be administered to one or both eyes of a subject. The CaSR antagonists and pharmaceutical compositions may be delivered by any mechanism, including to the ocular surface (e.g., as eye drops or ointment) or into the eyes (e.g., via punctal plug or subconjunctival injection). In some embodiments, the CaSR antagonists and pharmaceutical compositions is administered topically to the ocular surface. In more specific embodiments, the topical administration is topical administration or injection to the conjunctiva of the eye. In embodiments, the topical administration is topical administration or injection to the conjunctival sac of the eye. In embodiments, the topical administration is topical administration to the conjunctiva of the eye and the conjunctival sac of the eye. The active agents and compositions described herein can be delivered topically as a liquid formulation, e.g., as eye drops. In embodiments, the topical liquid formulation is a solution. In embodiments, the topical liquid formulation is an aqueous solution. In embodiments, the topical liquid formulation is a suspension. In embodiments, the topical liquid formulation is an emulsion.

In other embodiments, the CaSR antagonists and compositions described herein can be delivered through an implantable device in the eyes, including fluid-eluting contact lens. The implantable device includes a reservoir containing the pharmaceutical composition described herein and may further include means that allows the active agent to elute onto the ocular surface in a sustained manner. See, e.g., U.S. 2020/0409177.

In certain embodiments, the ophthalmically acceptable excipient may be water, NaCl, normal saline solutions, lactated Ringer's solution, sucrose, glucose, binders, fillers, disintegrants, lubricants, coatings, sweeteners, flavors, salt solutions, alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with other pharmaceutically acceptable excipients such as lubricants, preservatives, stabilizers, wetting agents, emulsifiers, salts for influencing osmotic pressure, buffers, coloring, and/or aromatic substances and the like.

### EXAMPLES

### Chemicals and Solutions

The compositions of perfusion solutions follow the solutions used in the standardized human nasal and ocular potential difference protocol.^{12,13} Solutions #1-3 (high Cl⁻, amiloride, and low Cl⁻, respectively) were made in 1-liter batches, pH balanced to 7.4, and filtered in a sterile environment prior to refrigeration and used within 3 months. Solution #1 (high Cl⁻) contained 1 L Ringer's injection (containing 147 mM NaCl, 2 mM CaCl₂, 4 mM KCl, 2.4 mM K₂HPO₄, 0.4 mM KH₂PO₄, and 1.2 mM MgCl₂. 100 μM amiloride was added to Solution #1 to create Solution #2 (amiloride). Solution #3 (low Cl⁻) was identical to Solution #1, except NaCl was replaced with sodium gluconate. Cinacalcet (CaSR agonist) and NPS-2143 (CaSR antagonist) were purchased from Tocris Bioscience (Minneapolis, MN, USA). All other chemicals were purchased from Sigma-Aldrich (St. Louis, MO, USA).

### Animals

BALB/c mice (female and male, 8-12 weeks old) were bred in UCSF Laboratory Animal Resource Center. The experimental protocols were approved by the UCSF Institutional Animal Care and Use Committee. Animal experiments were done in adherence with the ARVO Statement for the Use of Animals in Ophthalmic and Vision Research.

### Ocular Surface Potential Difference (OSPD) Measurements

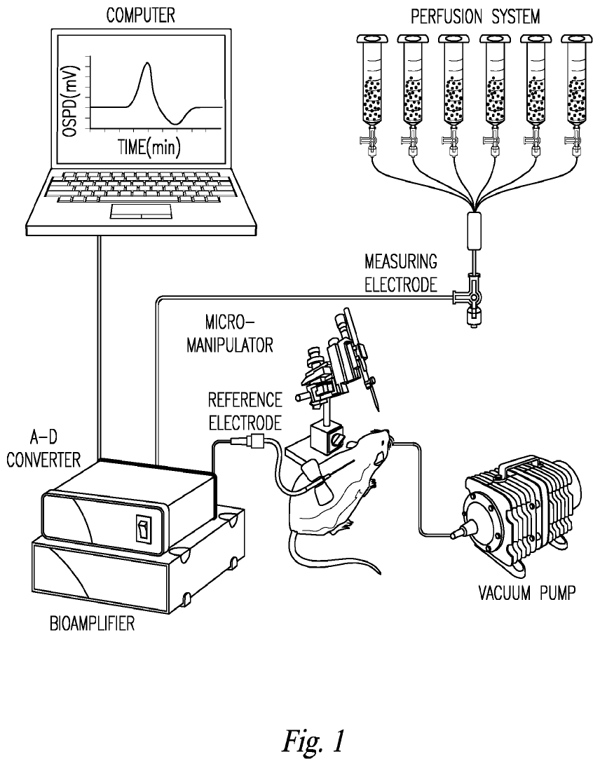

Ocular surface potential difference (OSPD) and tear fluid volume measurements were performed in mice with topically applied cinacalcet (CaSR activator) and NPS-2143 (CaSR inhibitor). Mice were anesthetized with isoflurane and body temperature was maintained at 37° C. via a heating pad. As described previously,^{12, 14, 15} open-circuit transepithelial potential differences were measured in response to serial perfusions of different isoosmolar (310 mOsm/kg H₂O) solutions over the ocular surface at 5-10 ml/min. The measuring electrode was in contact with the perfusion catheter which was carefully positioned using a three-axis micromanipulator directly above the mouse ocular surface and the reference electrode was grounded via a 23-gauge butterfly needle agar bridge inserted subcutaneously in the back. Measuring and reference electrodes consisted of Ag/AgCl with 3 M KCl agar bridges, and both electrodes were connected to an ISO-Z headstage, BMA-200 high-impedance amplifier/voltmeter, and PowerLab analog-to-digital converter (ADInstruments; Colorado Springs, CO, USA) connected to a computer (FIG. 1). Each solution was perfused onto the ocular surface using a gravity perfusion system (ALA Scientific: Farmingdale, NY, USA) for 1-3 minutes until a stable OSPD reading was obtained.

### Immunofluorescence Staining

CaSR immunostaining was performed in mouse and human cornea and conjunctiva. Sections were obtained from BALB/c mouse eyes and from human donors with no history of visual or ocular disease. Mouse and human eyes were imbedded and sectioned into 15 μm cryosections using a LEICA CM1860 (Leica Biosystems; Deer Park, IL, USA) and mounted onto Fisherbrand Superfrost Plus Microscope Slides (Fisher Scientific; Hampton, NH, USA). Sections were stored at −80° C. For immunostaining, sections were brought to room temperature, washed with phosphate-buffered saline (PBS) three times (5 minutes each) and incubated with block containing 50 mM Tris pH 7.4, 100 mM NaCl, 0.1% TX100, 3% Normal Goat Serum, 0.1% BSA, and deionized water for 1 hour at room temperature in a humidity chamber. Eye sections were then treated with Anti-CASR antibody [5C10, ADD](Abcam; Cambridge, UK) overnight at 4° C. The following day, slides were washed in washing solution containing 50 mM Tris pH 7.4, 100 mM NaCl, 0.1% TX100, and deionized water 4 times for 5 minutes each. Secondary antibody (Mouse: Cy3 AffiniPure Goat Anti-Mouse IgG2a: Human: Alexa Fluor 488 IgG2a Goat Anti-Mouse (γ2a); Fisher Scientific) was dissolved in block (1:500) and added for 2 hours at room temperature in a humidity chamber. Slides were again washed 4 times in washing solution for 5 minutes and once in PBS for 5 minutes. Sections were incubated for 5 minutes with Hoechst 33258 (nucleus marker) and then rinsed in deionized water. Slides were mounted with Fluoromount-G (Fisher Scientific) and covered with Corning Cover Glass 24 mm×50 mm (Corning Inc; Corning, NY, USA).

Controls were obtained using secondary antibody in the absence of primary antibody which confirmed no immunofluorescence in this setting.

### Tear Fluid Volume Measurements in Mice

Endodontic absorbent paper point tear tests (EAPPTT) were used to measure tear volume in BALB/c mice at baseline and 15 minutes, 1 hour, 3 hours, and 6 hours after application of a single 10 μL drop of PBS with 30 μM NPS-2143 (or 0.2% DMSO control) in both eyes. Awake, unanesthetized mice were gently immobilized and forceps were used to place the EAPPTT (Absorbent Points, size 30, standard 0.02 taper; Densply Maillefer; Tulsa, OK, USA) into the inferior conjunctival fornix for 20 seconds. The wetted length was measured using a millimeter-scale ruler viewed under a magnifying glass. All mice were kept at ambient conditions (approximately 50% humidity and 21° C.) and provided unrestricted access to food and water.

### Statistical Analysis

Experiments with two groups were analyzed using two-tailed unpaired Student's t-test; for three or more groups, analysis was done with one-way analysis of variance (ANOVA) and post hoc Newman-Keuls multiple comparisons test using GraphPad Prism (GraphPad Software: Boston. MA, USA). In all analyses, p<0.05 was considered as statistically significant.

### REFERENCES

1. Dartt D A, Willcox M D. Complexity of the tear film: importance in homeostasis and dysfunction during disease. Exp Eye Res. 2013; 117:1-3.
2. de Paiva C S. Effects of Aging in Dry Eye. Int Ophthalmol Clin. 2017; 57(2):47-64.
3. Kitazawa K, Inomata T, Shih K, et al. Impact of aging on the pathophysiology of dry eye disease: A systematic review and meta-analysis. Ocul Surf 2022; 25:108-118.
4. Dartt D A. Regulation of mucin and fluid secretion by conjunctival epithelial cells. Prog Retin Eye Res. 2002; 21(6):555-576.
5. Lindgren E S, Cil O, Verkman A S, Pasricha N D. Ocular Surface Ion Transport and Dry Eye Disease. Current Ophthalmology Reports. 2022; 10(4):188-197.
6. Ashok N. Khamar P, D'Souza S, et al. Ion channels in dry eye disease. Indian J Ophthahnol. 2023; 71(4):1215-1226.
7. Chen R A, Goodman W G. Role of the calcium-sensing receptor in parathyroid gland physiology. Am J Physiol Renal Physiol. 2004; 286(6):F 1005-1011.
8. Riccardi D, Valenti G. Localization and function of the renal calcium-sensing receptor. Nat Rev Nephrol. 2016; 12(7):414-425.
9. Nemeth E F, Goodman W G. Calcimimetic and Calcilytic Drugs: Feats, Flops, and Futures. Calcif Tissue Int. 2016; 98(4):341-358.
10. Oak A A, Chhetri P D, Rivera A A, Verkman A S, Cil O. Repurposing calcium-sensing receptor agonist cinacalcet for treatment of CFTR-mediated secretory diarrheas. JCI Insight. 2021; 6(4).
11. Oak A A, Chu T, Yottasan P, et al. Lubiprostone is non-selective activator of cAMP-gated ion channels and Clc-2 has a minor role in its prosecretory effect in intestinal epithelial cells. Mol Pharmacol. 2022; 102(2):106-115.
12. Pasricha N D, Smith A J, Levin M H, Schallhorn J M, Verkman A S. Ocular Surface Potential Difference Measured in Human Subjects to Study Ocular Surface Ion Transport. Transl Vis Sci Technol. 2020; 9(11):20.
13. Solomon G M, Bronsveld I, Hayes K, et al. Standardized Measurement of Nasal Membrane Transepithelial Potential Difference (NPD). Journal of Visualized Experiments. 2018(139).
14. Levin M H, Verkman A S. CFTR-Regulated Chloride Transport at the Ocular Surface in Living Mice Measured by Potential Differences. Investigative Opthalmologv & Visual Science. 2005; 46(4):1428.
15. Levin M H, Kim J K, Hu J, Verkman A S. Potential Difference Measurements of Ocular Surface Na+Absorption Analyzed Using an Electrokinetic Model. Investigative Opthalmnology & Visual Science. 2006; 47(1):306.
16. Arige V, Yule D I. Spatial and temporal crosstalk between the cAMP and Ca(2+) signaling systems. Biochim Biophys Acta Mol Cell Res. 2022; 1869(9):119293.
17. Tseng R C, Chen C C, Hsu S M, Chuang H S. Contact-Lens Biosensors. Sensors (Basel). 2018; 18(8).
18. Craig J P, Nelson J D, Azar D T, et al. TFOS DEWS II Report Executive Summary. Ocul Surf 2017:15(4):802-812.
19. de Paiva C S, Pflugfelder S C, Ng S M, Akpek E K. Topical cyclosporine A therapy for dry eye syndrome. Cochrane Database Syst Rev. 2019; 9(9):Cd010051.
20. Lollett I V, Galor A. Dry eye syndrome: developments and lifitegrast in perspective. Clin Ophthalmol. 2018; 12:125-139.
21. Kim M, Lee Y, Mehra D, Sabater A L, Galor A. Dry eye: why artificial tears are not always the answer. BMJ Open Ophthalmol. 2021; 6(1):e000697.
22. Flores A M, Casey S D, Felix C M, Phuan P W, Verkman A S, Levin M H. Small-molecule CFTR activators increase tear secretion and prevent experimental dry eve disease. The FASEB. Journal. 2016; 30(5):1789-1797.
23. Felix C M, Lee S, Levin M H, Verkman A S. Pro-Secretory Activity and Pharmacology in Rabbits of an Aminophenyl-1,3,5-Triazine CFTR Activator for Dry Eye Disorders. Investigative Ophthalmology & Visual Science. 2017:58(11):4506.
24. Lee S, Phuan P-W, Felix C M, Tan J-A, Levin M H, Verkman A S. Nanomolar-Potency Aminophenyl-1,3,5-triazine Activators of the Cystic Fibrosis Transmembrane Conductance Regulator (CFTR) Chloride Channel for Prosecretory Therapy of Dry Eye Diseases. Journal of Medicinal Chemistry. 2017; 60(3):1210-1218.
25. Chen X. Lee S, Zhang T, et al. Nanomolar Potency Aminophenyltriazine CFTR Activator Reverses Corneal Epithelial Injury in a Mouse Model of Dry Eye. Journal of Ocular Pharmacology and Therapeutics. 2020; 36(3):147-153.
26. Hori Y, Oka K. Inai M. Efficacy and Safety of the Long-Acting Diquafosol Ophthalmic Solution DE-089C in Patients with Dry Eye: A Randomized, Double-Masked. Placebo-Controlled Phase 3 Study. Adv Ther. 2022; 39(8):3654-3667.
27. Yarova P L, Huang P, Schepelmann M W, et al. Characterization of Negative Allosteric Modulators of the Calcium-Sensing Receptor for Repurposing as a Treatment of Asthma. J Pharmacol Exp Ther. 2021; 376(1):51-63.
28. Jiang P, Huang R, Ma N, Jiang F. The Expression of Calcium Sensing Receptor in Normal and Diabetic Rat Eves. Med Sci Monit. 2018; 24:706-710.
29. Chattopadhyay N, Ye C, Singh D P, et al. Expression of extracellular calcium-sensing receptor by human lens epithelial cells. Biochemn Biophvs Res Commnun. 1997:233(3):801-805.
30. Shinde V, Hu N, Mahale A, et al. RNA sequencing of corneas from two keratoconus patient groups identifies potential biomarkers and decreased NRF2-antioxidant responses. Sci Rep. 2020:10(1):9907.
31. Hough T A, Bogani D, Cheeseman M T, et al. Activating calcium-sensing receptor mutation in the mouse is associated with cataracts and ectopic calcification. Proc Natl Acad Sci USA. 2004; 101(37):13566-13571.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide vet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 63/507,430 filed Jun. 9, 2023, the entirety of which is incorporated by reference herein.

1. A method for treating ocular surface disorders in a subject in need thereof, the method comprising administrating to the subject a pharmaceutical composition comprising a calcium sensitive receptor (CaSR) antagonist and an ophthalmically acceptable excipient.

2. The method of claim 1 wherein the pharmaceutical composition is applied to an eye of the subject.

3. The pharmaceutical method of claim 2 wherein the pharmaceutical composition is applied to an eye of the subject every 4-12 hours, or preferably, every 6-8 hours.

4. The method of claim 1, wherein the ocular surface disorder is one or more dye eye diseases, keratoconjunctivitis, keratitis, or Sjogren's syndrome.

5. The method of claim 1, wherein the pharmaceutical composition is applied as eye drops, punctal plug, subconjunctival injectable, or through fluid-eluting contact lens.

6. The method of claim 1, wherein the CaSR antagonist has an IC₅₀ value of 10 μM or less.

7. The method of claim 1, wherein the CaSR antagonist is 2-chloro-6-[(2R)-3-([1,1-dimethyl-2-(2-naphthalenyl)ethyl]amino)-2-hydroxypropoxy]benzonitrile (NPS-2143), ronacaleret, encaleret NPS-2390, NPSP-795 (or SB-423562), calcium-sensing receptor antagonists I, Calhex 231, ligustroflavone, SB-423557, or CaSR antagonist-1.

8. A method for increasing tear production in a subject in need thereof, the method comprising administrating to the subject a pharmaceutical composition comprising a calcium sensitive receptor (CaSR) antagonist and an ophthalmically acceptable excipient.

9. The method of claim 8, wherein the pharmaceutical composition is applied to an eye of the subject.

10. The method of claim 8 wherein the pharmaceutical composition is applied to an eye of the subject every 4-12 hours, or preferably, every 6-8 hours.

11. The method of claim 8, wherein the CaSR antagonist has an IC₅₀ value of 10 μM or less.

12. The method of claim 11 wherein the CaSR antagonist is 2-chloro-6-[(2R)-3-([1,1-dimethyl-2-(2-naphthalenyl)ethyl]amino)-2-hydroxypropoxy]benzonitrile (NPS-2143), ronacaleret, encaleret NPS-2390, NPSP-795 (or SB-423562), calcium-sensing receptor antagonists I, Calhex 231, ligustroflavone, SB-423557, or CaSR antagonist-1.

13. A topical ophthalmic formulation for use in treating ocular surface disorders or increasing tear production, the ophthalmic formulation comprising a calcium sensitive receptor (CaSR) antagonist and an ophthalmically acceptable excipient.

14. The topical ophthalmic formulation of claim 13, wherein the ocular surface disorders include dry eye diseases, keratoconjunctivitis, keratitis, or Sjogren's syndrome.

15. The topical ophthalmic formulation of claim 13, wherein the CaSR antagonist has an IC₅₀ value of 10 μM or less, and preferably, 2-chloro-6-[(2R)-3-([1,1-dimethyl-2-(2-naphthalenyl)ethyl]amino)-2-hydroxypropoxy]benzonitrile (NPS-2143), ronacaleret, encaleret NPS-2390, NPSP-795 (or SB-423562), calcium-sensing receptor antagonists I, Calhex 231, ligustroflavone, SB-423557, or CaSR antagonist-1.
